(19) United States
(12) Patent Application Publication
(10) Pub. No.: US 2009/0181007 A1
(43) Pub. Date: Jul. 16, 2009

(54) CULTURE MEDIUM AND PHARMACEUTICAL COMPOSITION FOR REGENERATING CARTILAGE TISSUE, A METHOD, USES AND PRODUCTS RELATED THERETO

(76) Inventors: Luisa Gennero, Torino (IT); Antonio Ponzetto, Moncalier (Torino) (IT); Enrico De Vivo, Venaria Reale (Torino) (IT); Luciano Contu, Alice Castello (Vercelli) (IT); Emanuella Morra, Torino (IT); Chiara Cesano, Torino (IT); Giovanni Cavallo, Torino (IT); Giuseppe Cicero, Torino (IT); Gian Piero Pescarmona, Torino (IT); Tetyana Denysenko, Torino (IT); Maria Augusta Roos, Torino (IT); Maurizio Sara, Moncalieri (Torino) (IT)

Correspondence Address: Steinfl & Bruno, 301 N Lake Ave Ste 810, Pasadena, CA, 91101, US

(21) Appl. No.: 12/296,850

(22) PCT Filed: Apr. 12, 2007
(86) PCT No.: PCT/IB2007/051325

§ 371 Date: Oct. 10, 2008

(30) Foreign Application Priority Data
Apr. 14, 2006 (IT) TO2006A000282

(51) Int. Cl. A61K 38/48; C12N 5/00; C12N 5/02; C12N 9/00; C12N 9/50; C12N 9/48; C12N 9/22; C12N 9/66; C12N 9/68; C12N 9/52; C12N 9/62; A61K 38/46; A61P 43/00

(52) U.S. Cl. 424/94.64; 435/377; 435/405; 435/325; 435/183; 424/94.63; 435/395; 435/219; 435/212; 435/199; 435/218; 435/217; 435/220; 435/225; 424/94.6; 424/94.65; 424/94.67

(57) ABSTRACT

A composition for in vitro use as a culture medium or in vivo use as a pharmaceutical composition or a medical device, capable of accelerating the differentiation of stem cells into cells with a chondrocytic phenotype and of restoring the original trophism of chondrocytes, is described. The composition comprises, in combination, at least one proteolytic enzyme, at least one growth factor and at least one from a sugar, an amino acid, a vitamin factor, a vitamin, a nucleotide and a nucleoside, in a physiologically acceptable carrier or diluent. A method of differentiating stem cells in cells having a chondrocytic phenotype, the cells obtained by the method and their uses, for example in human or animal cell therapy, for example by CBMP (Cellular Based Medicinal products) are also described.

The present invention relates to a culture medium, a pharmaceutical composition, a medical device, the method related thereto and the uses and products related thereto, for stimulating the growth of cartilage tissue. In particular, the object of the present invention is to stimulate differentiation of pluripotent stem cells into cartilage tissue, regeneration of cartilage tissue, as well as improving the viability of articular cartilages, even in compromised sites, by inducing regeneration of chondrocytes from native and non-native pluripotent stem cells.

Cartilage tissue, or cartilage, is a specialised form of connective tissue characterized by support functions and resistance to friction or external stress. Said tissue is constituted by cells designated as chondrocytes, which are surrounded by an intercellular substance with fibres immersed in an amorphous, compact gel-phase matrix.

Cartilage is neither vascularised nor innervated and exists in three distinct types
hyaline cartilage,
fibrous cartilage,
elastic cartilage.

Cartilage is covered by dense, vascularised connective tissue, the perichondrium, which is missing in both hyaline articular or “incrustation” cartilage and fibrous cartilage.

The perichondrium consists of two layers: an outer layer, which constitutes an actual connective tissue coating capsule (dense, fibrillar connective tissue), and a more inner layer formed by cells included in a reticulate-like connective tissue mesh; such cells are labeled chondrogenic since they are capable of division, giving rise to novel cartilage cells.

Articular cartilage surrounds the bone of moving joints (diarthrodial joints) helping the movement of the bone surfaces, and is devoid of perichondrium, which could hinder such movement. Nutrients are carried to articular cartilage by the synovial fluid, produced by the synovia. Synovial fluid also acts as a lubricant.

Hyaline cartilage derives its name from its translucent vitreous appearance, and is the most abundant form in the body. In adults, it is found in such locations as the sternocostal joints, on the articular surfaces of bones, the growth cartilage of long bones, tracheal rings, the large bronchi, in the cartilage structures of the nose and parts of the laryngeal cartilage. Like all connective tissue, hyaline cartilage is constituted by cells immersed in an extra-cellular matrix consisting of fibres and amorphous fundamental substances.

Fibrous and elastic cartilages are variants of hyaline cartilage, in which there is a predominance of collagen fibres or elastin fibres respectively. Fibrous cartilage is found near the tendons and ligament insertions; it is similar to dense fibrillar connective tissue, but containing chondrocytes in place of the fibroblasts. Elastic cartilage is found in nasal and laryngeal (epiglottis) cartilage and the auricle.

The cartilage matrix is a dense gel, but as it is not mineralized like bone tissue, it allows the diffusion of nutrients from the vessels found at the edge of the cartilage itself.

Cartilage cells are chondroblasts and chondrocytes.

The use of molecules capable of stimulating the proliferation and differentiation of stem cells into chondrocytes is well known [1-5]. Such substances are generally constituted by collagen, proteoglycans (for reference, see US-A-2004/0082623), aminoacids (as described in WO-A-03/050250; U.S. Pat. No. 6,596,274; US-A-2004/0151709), vitamins (see WO-A-03/024463; US-A-2001/0012965; U.S. Pat. No. 6,365,405), hyaluronic acid, sugars (for example US-A-2003/0026786; US-A-2004/0235165), linolenic/arachidonic acid (see US-A-2005/0118714; US-A-2003/0175964), as well as growth factors, such as for example fibroblast growth factor, epidermal growth factor, insulin-like growth factor, ITS (Insulin-Tranferrin-Selenium), transforming growth factor beta (TGF-beta), and others (as described in WO-A-97/18299 WO-A-00/17321; EP-A-1 077 253; U.S. Pat. No. 5,962,325; U.S. Pat. No. 6,150,163; US-A-2002/0115647; WO-A-2004/083415; WO-A-2004/093934; US-A-2004/0137612; US-A-2005/0090002) or also peptides or various proteins (for reference, see EP-A-1 441 028; U.S. Pat. No. 6,464,983; U.S. Pat. No. 6,610,656).

The use of matrices of various kinds and compositions into which are introduced stem cells for their differentiation into chondrocytes, and then replication of the chondrocytes with a defined three-dimensional structure, is also well known. The substrates normally used are constituted by polymers of various kinds (layers, rather than pellets) and various compositions (such as alginates, hydroxyapatite, collagen structures, polyglycolic acid-based polymers and others). U.S. Pat. No. 6,637,437 and US-A-2004/0214322 are cited by way of example.

### An Outline of Pathologies of Cartilage Tissue

Osteo-articular disorders affect over 17% of Italians (data from ISTAT 2001), and this is expected to increase in the future, in parallel with the increasing life expectancy of the populations of Italy, Europe and the industrialized countries in general (70% of the population of the United States aged over 65 and 42% of the European population). Disorders of the joint effect movement, whether of the sacro-iliac joint, or the tibio-femoral joint, or the vertebral column; consequently, they have a heavy impact on social and family life, particularly in older individuals. However, joint disorders also occur frequently in working age, both arthritis (juvenile rheumatoid arthritis, reactive arthritis, infective arthritis) and as a consequence of work or road-related trauma. Joint disorders alter the capacity of movement and physical appearance (deformity, gibbosity), and in any case affect personal and social life.

Disorders of the joint cartilage can affect individuals of any age, but are particularly important when they occur in young patients leading an active life.

It is well known that, unlike bone tissue, which is endowed with great regenerative capacity, hyaline cartilage is characterized by the absence of a blood, lymphatic and nervous support, indispensable for tissue repair. Indeed, only small losses of mass are replaced by fibro-cartilage tissue, while larger losses are rarely filled in: lesions of a certain depth (Outerbridge grade III-IV) have no possibility of healing spontaneously, and, in the long-term progress towards degeneration of the joint surface.

The rheumatic pathologies can be distinguished in three major groups: inflammatory, extra-articular and degenerative rheumatisms. The latter category is the most frequent (arthrosis).

Rheumatoid polyarthritis, often simply referred to as arthritis, ankylosing spondyloarthritis and collagenosis all fall within the group of inflammatory rheumatisms or arthritis.

The extra-articular rheumatisms do not affect the joints. This includes for example, tendonitis and fibromyalgia. The latter is a chronic disease, characterized by generalized and widespread pain, accompanied by profound fatigue.

The term “degenerative rheumatisms” or arthrosis is intended to indicate a non-inflammatory disorder resulting in the degeneration of one or more joints and affecting the knee joints, hip joints, the fingers and the spinal column. Said disorder results from the progressive alteration of the cartilage surrounding the ends of the bones. In arthritis, the cartilage, which acts as a shock-absorbing pad allowing the joints to move freely, deteriorates, so that the surface becomes coarse. Once degraded, cartilage is incapable of regenerating completely, and so dampening the impact resulting from movement. With passing time, the bone ends become increasingly less smooth. Joint deformations can occur, and the bones can assume incorrect positions. In this situation, the tendons and muscles are subjected to unusual stress, causing overloading, pain, stiffness and reduced mobility. Spasmodic (i.e. with acute phases) inflammation can also occur: this results in cartilage fragments inside the joint cavity, with excess fluid production and intra-articular effusion.

Chondropathy is distress of the cartilage tissue (it is referred to as chondromalacia when there is an ongoing degenerative process).

Recurring polychondritis is an episodic and destructive inflammatory disorder, affecting cartilage and other connective tissues, including the ear, joints, nose, larynx, trachea, eye, heart valves, kidney and blood vessels.

Chondroma is a benign tumour derived from cartilage tissue. It begins in segments of the skeleton, and is referred to as enchondroma when it develops deep in a bone, and ecchondroma when it develops outwardly.

Chondromatosis is a disease of unknown origin, characterized by the presence of cartilage masses inside the bone. Through growth, such masses can form tumors, which can be of various sizes, and can furthermore cause disturbed growth in the skeletal segments affected.

Another pathology of cartilage tissue is multiple chondromatosis. The sites most often affected are the tubular bones of the hands and feet, and the metaphysis of the long bones. These are chondromas which develop inside the bone.

Other pathologies include Ollier's disease or enchondromatosis (this is a genetically inherited deforming condition, which appears around the age of two with cartilage tumours of various sizes and progressive growth, localized in a number of bones) and chondrosarcoma (malign tumor derived from cartilage tissue, frequently localized to the pelvis, femur or humerus).

### The Therapeutic Treatments for Pathologies of Cartilage Tissue

Lesions of the joint cartilage are very frequently occurring events which can cause chronic pain, joint restriction and progressive arthritic degeneration.

Cartilage is not a vascularized tissue (i.e. does not contain blood vessels). A delicate equilibrium exists between its protein, glycoprotein (chondroitin and proteoglycans) components and water. Cartilage binds water and the nutrients it requires during joint movement. By looking after the joints and making them move correctly (and constantly), it is possible, within certain limits, to prevent wear of the cartilage and ensure regeneration. However, to promote this process, it is essential to provide cartilage tissue with the necessary nutrients.

To date, the surgical procedures available for the treatment of arthrosis are manifold, but differ depending on the type of lesion and the age of the patient. Again in this case, the indication is the determining factor. The surgical procedures currently used include techniques without implanting materials, such as debridement, abrasion chondroplasty and/or subchondral perforation or microfracture, with the aim of stimulating the formation of repair tissue. Such a tissue is comprised of fibro-cartilage reacting to reparative stimuli. In particular, fibro-cartilage possesses mechanical characteristics which are different from those of hyaline cartilage, in particular, lower load strength at the joint, thus this technique may be used for mild chondropathies or in severely degenerative clinical situations where no other type of treatment is possible. For more substantial losses of cartilage substance, techniques involving the implantation of material are used, such as perichondrial or periosteal implants, autologous osteochondral tissue mosaic transplants, autologous chondrocyte transplants, up to massive autologous or homologous osteochondral transplants in the case of loss of both bone and cartilage from the articular surface.

Autologous chondrocyte transplant is the therapy of choice for the treatment of large cartilage tissue losses.

Implanting autologous chondrocytes with a hyaluronic acid support offers a simplified way of applying the cells into the defect, while avoiding the use of a periosteal flap, the use of which is obligatory when the chondrocytes are brought in aqueous suspension, allowing the procedure to be performed by arthroscopy. Furthermore, it was shown that the use of three-dimensional support structures (“scaffolds”) promotes maintenance of the chondrocytic phenotype and the production of cartilage extra-cellular matrix under in vitro and in vivo culture conditions.

### Three-Dimensional Support Structures (“Scaffolds”)

The term “scaffold” is understood as meaning a porous, three-dimensional support made from a biocompatible and bioerodible material onto which the cells initially adhere and subsequently regrow until forming the tissue, in such a way that it biodegrades at a rate similar to that of regrowth. Over the course of various studies, a number of materials were identified which allowed the major characteristics of the aforementioned supports to be defined [5].

1. Collagen; this is a natural component of mammalian tissues, and when polymerized into three-dimensional supports, promotes and sustains cell adhesion in vitro by mimicking, in its overlapping layers, the original tissue.
2. Fibrin; this is the basic protein involved in the blood clot formation process. When polymerized into three-dimensional supports, fibrin promotes cell adhesion in vitro.
3. Alginates; these are polysaccharides derived from marine algae. They form hydrogel solutions, and have the advantage of allowing the uniform distribution of seeded cells and provide a support for their three-dimensional growth.
4. Hyaluronic acid or hyaluronan; this is a glycosaminoglycan sulphate and constitutes part of the cartilage matrix supporting the chondrocyte implant.
5. Polyglycolic acid (PGA); this is a polymer support largely used in the reconstructive engineering of cartilage tissue.
6. Hydroxyapatite; this is a bioceramic, chemically similar to the mineral component of mammalian bone.

### Surgical Treatments

To date, the object of the surgical treatments used was that of inducing the growth of repair tissue at the site of the defect by means of chondroplasty/chondroabrasion techniques, with or without subchondral perforation or microfracture. In every case, the fibro-cartilage repair tissue which forms has different biochemical and bioelastic characteristics from those if hyaline cartilage. The result obtained by means of such methods is merely a delay in the development of the progressive arthritic process. For such reasons, research has turned towards alternative solutions. Recently, innovations in the field of cell culture have allowed the ex vivo manipulation of autologous cells and their use for reparative surgery, without modifying their native phenotype and functionality. The possibility of transplanting autologous human chondrocytes for the reconstruction of deteriorated cartilage following repeated trauma or pathologies, such as osteochondritis dissecans and patellar chondromalacia, was demonstrated. The results obtained showed the formation of new cartilage, with characteristics entirely similar to hyaline cartilage, which expresses type-II collagen.

One of the criticisms aimed at the above-described method relates to the method of in situ implantation of the chondrocytes by means of injection under a disc of periosteum. Said process has raised doubts surrounding the fact that cartilage reconstruction can be induced by periosteal mesenchymal cells and not by the injected autologous chondrocytes. Studies conducted in animals with labeled cartilage cells have confirmed that tissue growth is also attributed to the transplanted cells.

### DESCRIPTION OF THE INVENTION

The object of the present invention is that of finding a valid and effective solution for stimulating the growth of cartilage tissue and improving the viability of joint cartilage, even in compromised sites, by the induction of regeneration of existing chondrocytes and differentiation of native and non-native pluripotent stem cells into chondrocytes.

According to the present invention, said object is achieved by the solution specifically claimed in the following claims. The claims form an integral part of the technical teachings provided herein in connection with the invention.

The invention relates to a composition which is capable of promoting and stimulating differentiation of pluripotent stem cells into chondrocytes as well as the proliferation of the chondrocytes themselves, or the growth of cartilage tissue, and which is capable of improving the viability of the cartilage thereby produced.

The present invention is based on the observation of the particular proliferative stimulus exercised by certain proteolytic agents on chondrocytes and on pluripotent stem cells. Such observations lead to the formulation of a composition for in vitro use as a culture medium, or in vivo use as a pharmaceutical composition, which is capable of inducing differentiation of pluripotent stem cells into cartilage tissue, of stimulating the growth of cartilage tissue and of improving the viability of cartilage tissue and joint cartilage. Compromised chondrocytes regain viability and actively deposit matrix, even after 3 months of in vitro culture. The present study confirms the efficacy of a culture procedure for autologous chondrocytes. The in vitro histological results obtained after 6 months of culture with the culture media of the present invention confirm the formation of hyaline cartilage morphologically comparable to undamaged cartilage in vivo, with optimal histo-functional characteristics.

### DETAILED DESCRIPTION OF THE INVENTION

Purely by way of non-limiting example, the invention will now be described in detail with reference to certain particularly preferred embodiments.

The different culture media (for example C-BASE and C-BASE PLUS illustrated in tables 1 and 2) and the pharmaceutical composition (for example C-BASE INFUS, illustrated in table 4) of the invention turned out to be effective in inducing differentiation towards a normally-functioning chondrocytic phenotype in mesenchymal cells derived from bone marrow, and in cells isolated from synovial fluid and membranes, as well as in inducing differentiation towards a normally-functioning chondrocytic phenotype in pluripotent monocytes isolated from peripheral blood and in monocytoid cells lines. For this purpose the monocytoid cell line PSC-THP1 (accession number ICLC PD No. 05005; deposited on 18 Oct. 2005; described in Italian patent application TO2005A000819), having a pluripotent stem-cell phenotype, and PSC-THP1-CHONDROCYTE-LIKE (accession number ICLC PD No. 06001; deposited on 21 Mar. 2006), having a stably induced chondrocytic phenotype, were used.

All the cartilage tissue biopsy samples tested responded positively to the use of the culture media of the present invention, remaining viable, depositing matrix and showing ordered, three-dimensional distribution during 1 month of culture in vitro. It should be noted that after 1 month of culture in vitro such tissues were frozen at −80° C. (10% DMSO+50% serum+40% culture medium) and then normally thawed out and reactivated in culture for a further 1 month, again ascertaining normal histo-functionality.

The viability of the atrophic chondrocytes appeared significantly improved after 15 days of treatment. Without wishing to be bound by any specific theory in that regard, the present inventors believe that the results obtained with the culture media of the present invention demonstrate that the state of cartilage atrophy that occurs in degenerative processes is recoverable.

In greater detail, the composition of the present invention comprises, in combination, at least one proteolytic enzyme, at least one of a sugar, a vitamin, a vitamin factor, an amino acid, a nucleotide and a nucleoside, in a physiologically acceptable carrier or diluent. The composition may additionally comprise as a growth stimulating factor a parasympathicolytic (preferably scopolamine) and/or a corticosteroid (preferably desametasone) and/or other ingredients described in further detail hereinafter.

The composition of the present invention may be in the form of a culture medium or pharmaceutical composition. The composition of the invention has demonstrated evidence of excellent growth and development of cartilage tissue with normal histo-functional characteristics in vitro. This is demonstrated by the culture of cartilage tissue biopsies and chondrocytes; by the induction of a normally-functioning chondrocytic phenotype in monocytes isolated from peripheral blood and in monocytoid cell lines (preferably THP-1 lines, more preferably, the cell line PSC-THP1 (ICLC PD No. 05005 deposited on 18 Oct. 2005) having a pluripotent stem cell phenotype, and even more preferably the cell line PSC-THP1-CHONDROCYTE-LIKE (ICLC PD No. 06001 deposited on 21 Mar. 2006) having a stably induced chondrocyte phenotype), as well as by the induction of a normally functioning chondrocytic phenotype in mesenchymal cells derived from bone marrow and in cells isolated from synovial fluid and membranes.

Alternatively, the composition of the invention is a composition as defined above but devoid of any proteolytic enzyme or growth factor, and it is intended to be administered to a pathological site characterized by the presence of proteolytic enzyme-producing pro-inflammatory cells. The proteolytic enzymes produced in situ by the pro-inflammatory cells, helped by the components of the administered composition, are capable of exerting a proliferating stimulus on the chondrocytes and the pluripotent stem cells as mentioned above. The composition according to this alternative embodiment is suitable for use as a medical device. Table 3 shows a composition according to this embodiment, designated as C-BASE-INFUS-MD.

The functions exercised by the composition of the present invention are mainly the following:
The composition in the form of the culture medium C-BASE possesses the characteristics and the ability to stimulate the growth of cartilage tissue through direct action on chondrocytes with the recovery of atrophic tissues. All the atrophic cartilage tissue biopsies treated in vitro appear re-invigorated and show active matrix deposition, which was previously lacking. C-BASE solution lends itself to therapeutic use in the form of its specific composition for infiltrations (C-BASE-INFUS) in arthroscopy or intra-articular infusion, allowing the restoration of moderately compromised cartilage.
The pharmaceutical composition C-BASE-INFUS, the medical device C-BASE-INFUS-MD and the culture medium C-BASE PLUS, variants of the C-BASE solution, enhance the differentiation of pluripotent stem cells into chondrocytes and into normally histo-functioning cartilage tissue.
The culture media and the pharmaceutical composition according to the present invention can be advantageously used with fibrin sponges, collagen membranes, hyaluronic acid supports or any of the supports previously described.
Suspensions of pluripotent stem cells induced to differentiate into chondrocytes by the use of the culture media according to the present invention can be used in relation to arthroscopically assisted transplantations.

The innovative nature of this research is expressed in a culture medium, a pharmaceutical composition and in the method related thereto in which nutrients (e.g. glucose, hyaluronic acid, amino acids, vitamins) and certain growth factors and a proteolytic agent (e.g. papain and/or endogenous proteolytic enzymes) were used for the first time along with growth factors, corticosteroids and/or parasympathicolytics for the recovery of atrophic chondrocytes and the induction of pluripotent stem cells into chondrocytes.

In the light of the foregoing, the method, culture media and pharmaceutical composition of the present invention can be advantageously used, for example, in orthopaedics, rheumatology, otorhinolaryngology, maxillo-facial surgery, odontostomatology, cardiology, dermatology, reconstructive and aesthetic surgery.

### Materials and Methods

The culture media and the pharmaceutical composition of the present invention were prepared using, inter alia, the substances listed below:

### 1. Amino Acids:

methionine, cystine, N-acetylcysteine, cysteine, glycine, leucine, isoleucine, proline, glutamine, arginine, glutamic acid, histidine, histidine-HCl, lysine, lysine-HCl, phenylalanine, serine, threonine, tryptophane, tyrosine, tyrosine-disodium salt, valine, proline, hydroxyproline, a solution containing all the non-essential aminoacids.

### 2. Peptides:

Glutathione, collagen, elastin, wheat extract, polypeptides to which trophic functions are attributed.

### 3. Vitamins:

Retinoic acid, retinol, ascorbic acid, pantothenic acid, D-calcium pantothenate, pyridoxine, pyridoxine-HCl, folic acid, niacinamide, riboflavin, cobalamine, para-aminobenzoic acid and biotin.

### 4. Vitamin Factors:

Inositol, myo-inositol, choline chloride, pyruvic acid, sodium pyruvate, putrescine and putrescine-HCl.

### 5. Growth Factors:

TGF-B (transforming growth factor Beta), LIF (leukaemia inhibitory factor), ITS (insulin-transferrin-selenium), insulin, M-CSF (macrophage colony stimulating factor), IL-2 (Interleukin-2), PMA (phorbol-12-myristate-13-acetate), autologous serum, corticosteroids (for example desametasone), parasympathicolytics (for example scopolamine), glucagon.

### 6. Salts:

Calcium gluconate, calcium phosphate, sodium bicarbonate, calcium chloride, magnesium chloride, magnesium sulphate, potassium chloride, potassium phosphate, sodium chloride, calcium nitrate, zinc chloride, ferric nitrate, sodium pyruvate, D-calcium pantothenate, tyrosine disodium salt.

### 7. Proteolytic Enzymes:

Papain, collagenase (preferably type-Ia, type-II, type IV), serratiopeptidase, heparanase, DNAse, elastase, bromelain, bradykinase, Clostridium peptidase, enzymes expressed by Lactobacillus acidophilus, enzymes expressed by the genus Aspergillus, proteases, aliinase, fibrinolysin.

### 8. Mucopolysaccharides:

Hyaluronic acid, chondroitin sulphates.

### 9. Sugars, Alcohols Derived Therefrom and Mixtures Thereof:

Glucose, sucrose, glucans, mannans, glucomannans, fucose, fructose, heparan sulphates, pectins, starches, the alcohols derived therefrom.

### 10. Cell Culture Solutions:

RPMI 1640 (cell culture medium), DMEM-LG (cell culture medium), FBS (foetal bovine serum for cell culture), F12 (cell culture solution containing a complete amino acid source), HANK'S solution (cell culture solution containing sodium bicarbonate).

### 11. Haemoderivatives:

Autologous serum prepared from the peripheral blood from tissue, cell and culture medium donors and recipients.

### Culture Media and Pharmaceutical Composition

The culture media of the present invention for in vitro use and the pharmaceutical composition or medical device for in vivo use, were prepared using the substances in the amounts indicated in tables 1 to 4.

For all of the formulations listed hereinafter, the substances were weighed-out as required by the formula, to give 1 liter of the final solution.

TABLE 1
Culture medium C-BASE

| Substance | Conc. mg/L | |
|---|---|---|
| Methionine | 4.94 | |
| Cystine | 5.12 | |
| N-acetylcysteine | 201.00 | |
| Cysteine | 3.51 | |
| Glycine | 4.37 | |
| Leucine | 15.90 | |
| Proline | 4.67 | |
| Glutamine | 45.00 | |
| Biotin | 0.055 | |
| Pantothenic acid | 0.25 | |
| Ascorbic acid | 515.00 | |
| Retinol | 0.0015 | |
| Papain | 0.20 | |
| Calcium Gluconate | 100 | |
| Hyaluronic acid | 0.50 | |
| Glucose | 2000.00 | |
| Solutions | L/sol. | |
| RPMI 1640 | q.b. to 1 liter of | |
| | solution | |
| FBS | 100 | mL |
| F12 | 20 | mL |
| HANK'S solution | 20 | mL |
| Autologous serum | 4 | mL |
| Insulin | 10 | units |
| Non-essential amino acids | 20 | mL |
| ITS | 8 | mL |
| Miscellaneous | weight/volume | |
| TGF-B | 20 | μg |
| LIF | 10 | μg |

TABLE 2
Culture medium C-BASE PLUS

| Substance | Conc. mg/L | |
|---|---|---|
| Methionine | 4.94 | |
| Cystine | 5.12 | |
| N-acetylcysteine | 1.00 | |
| Cysteine | 3.51 | |
| Glycine | 4.37 | |
| Leucine | 15.90 | |
| Proline | 4.67 | |
| Glutamine | 45.00 | |
| Biotin | 0.05 | |
| Pantothenic acid | 0.25 | |
| Ascorbic acid | 515.00 | |
| Retinol | 0.0015 | |
| Papain | 0.20 | |
| Hyaluronic acid | 0.50 | |
| Glucose | 2000.00 | |
| Solutions | L/sol. | |
| RPMI 1640 | q.b. to 1 liter of solution | |
| FBS | 100 | mL |
| F12 | 20 | mL |
| HANK'S solution | 20 | mL |
| Autologous serum | 4 | mL |
| TGF-B | 20 | μg |
| Insulin | 10 | units |
| Non-essential amino | 20 | mL |
| acids | | |
| LIF | 10 | μg |
| ITS | 8 | mL |
| Biotin | 2.5 | ng |
| N-acetyl-cysteine | 0.2 | g |
| Calcium Gluconate | 0.1 | g |
| Dexamethasone 21- | 40 | μg |
| phosphate disodium salt | | |

TABLE 3
Pharmaceutical composition C-BASE-INFUS-MD

| Substance | Conc. mg/L |
|---|---|
| Sterile deionized water | q.b. to 1 liter of solution |
| Ascorbic acid | 545.00 |
| Aspartic acid | 57.506 |
| Glutamic acid | 61.454 |
| Alanine | 25.1 |
| Arginine-HCl | 246.37 |
| Asparagine-H₂O | 87.1702 |
| Calcium Gluconate | 104.582 |
| Cystine-2HCl | 91.3756 |
| Choline Chloride | 3.2792 |
| D-Biotine | 0.350146 |
| Desametasone 21-disodium | 0.044 |
| phosphate | |
| Phenylalanine | 15.0992 |
| Glycine | 109.1502 |
| Glucose | 50000.00 (50 g/L) |
| Glutamine | 435.38 |
| Isoleucine | 50.0788 |
| Hystidine-HCl-H₂O | 20.601 |
| Leucine | 98.262 |
| Lysine-HCl | 40.73 |
| KCl | 412.48 |
| Methionine | 30.0896 |
| MgSO₄•7H₂O | 246.5 |
| Myoinositol | 35.36 |
| NaHCO₃ | 2030.52 |
| NaCl | 6311.98 |
| Na₂HPO₄ | 803.7984 |
| Proline | 66.89 |
| Scopolamine | 2 |
| Serine | 59.61 |
| Tyrosine-disodium salt | 25.018 |
| Threonine | 20.238 |
| Triptophane | 5.0408 |
| Valine | 117.234 |
| Vitamine A as retinoic acid | 0.0045 |
| ZnCl₂ | 0.00806 |

TABLE 4
Pharmaceutical composition C-BASE-INFUS

| Substance | Conc. mg/L | |
|---|---|---|
| CaCl₂•2H₂O | 4.582 | |
| MgCl | 1.15 | |
| MgSO₄•7H₂O | 246.5 | |
| KCl | 412.48 | |
| KH₂PO₄ | 1.2 | |
| NaHCO₃ | 2030.52 | |
| NaCl | 6311.98 | |
| Na₂HPO₄ | 803.7984 | |
| Ca(NO₃)₂•4H₂O | 100 | |
| ZnCl₂ | 0.00806 | |
| Fe(NO₃)₃ 9H₂O | 0.000002 | |
| Calcium Gluconate | 100 | |
| Arginine-HCl | 246.37 | |
| Cystine-2HCl | 91.3756 | |
| Glutamine | 435.38 | |
| Glutamic acid | 61.454 | |
| Glycine | 109.1502 | |
| Histidine-HCl-H₂O | 20.601 | |
| Lysine-HCl | 40.73 | |
| Isoleucine | 50.0788 | |
| Leucine | 98.262 | |
| Methionine | 30.0896 | |
| Phenylalanine | 15.0992 | |
| Serine | 59.61 | |
| Threonine | 20.238 | |
| Tryptophane | 5.0408 | |
| Tyrosine-disodium salt | 25.018 | |
| Valine | 117.234 | |
| Proline | 66.89 | |
| Aspartic acid | 57.506 | |
| Asparagine-H₂O | 87.1702 | |
| Alanine | 25.1 | |
| Hydroxyproline | 20 | |
| Myo-inositol | 35.36 | |
| Ascorbic acid | 545 | |
| Choline chloride | 3.2792 | |
| Folic acid | 1.0264 | |
| Niacinamide | 1.00074 | |
| D-Calcium Pantothenate | 1.0096 | |
| Pyridoxine-HCl | 1.00124 | |
| Riboflavin | 0.20076 | |
| D-Biotin | 0.350146 | |
| Para-aminobenzoic acid | 1 | |
| Sodium pyruvate | 2.2 | |
| Putrescine-HCl | 3.22 | |
| Cobalamine | 0.0322 | |
| Retinoic acid | 0.0045 | |
| Papain | 0.2 | |
| Hyaluronic acid | 1.5 | |
| Glucose | 50000 | |
| Glutathione (reduced) | 100 | |
| Dexamethasone 21- | 40 | μg/liter |
| phosphate disodium salt | | |
| Solutions | L/sol. | |
| Autologous serum | 2 | mL |

| | |
|---|---|
| Sterile deionised water | q.b. to 1 liter |
| | of solution |

### Pharmaceutical Composition C-BASE-INFUS: Operation Protocol.

In order to prevent anaphylactic shock following intra-articular infusion therapy, prior to the administration of C-BASE-INFUS, prophylactic treatment with antihistamines and/or cortisones, such as for example, diphenylhydramine, histamine type-I receptor antagonists, cetirizine, loratadine, fexofenadine, betamethasone-disodium-phosphate, hydrocortisone, methylprednisolone, can be administered.

### Cell Cultures

Isolation of Chondrocytes from Cartilage Tissue and Native Tissue Reconstitution In Vitro

Samples of cartilage and peripheral blood from the same patent are treated fresh (within 2 hours of explant). Peripheral blood from three tubes, with EDTA anticoagulant, are centrifuged at 160 g for 10 minutes at room temperature. The supernatant is removed and stored in 1 mL aliquots (storage at −20° C.).

The cartilage biopsies, removed using sterile forceps (in alcohol and then flamed), are placed in 100 mm diameter plates (Falcon, Becton Dickinson Labware Europe, Milan, Italy) and 10 mL of unsupplemented RPMI 1640 medium (Life Technologies, Grand Island, N.Y.) added. With the aid of sterile (in alcohol and then flamed) forceps and scissors, the samples are cut up further into small pieces, and washed in the plates twice. The samples are transferred to a 100 mm diameter plate (Falcon, Becton Dickinson Labware Europe, Milan, Italy), into which is prepared 10 mL of RPMI 1640 medium (Life Technologies, Grand Island, N.Y.), 200 μL gentamycin and 1 mg/mL collagenase. All samples were incubated for 2 hours in a Heraeus thermostatically controlled incubator at a temperature of 37° C. in an atmosphere containing a constant flow of 5% CO₂ (v/v in air). Following incubation, 4 mL of FBS (Foetal Bovine Serum) are added to the samples in order to quench the collagenase activity. The undissolved pieces and all the solution, with the cells in suspension, are collected and centrifuged at 160 g for 10 minutes at room temperature. The pellet is resuspended in unsupplemented RPMI 1640 medium (Life Technologies, Grand Island, N.Y.) and washed twice by means of centrifugation at 160 g for 10 minutes at room temperature. Pellets are resuspended in 5 mL of DMEM-LG medium (Gibco, Grand Island, N.Y.) supplemented with F12 solution (10% of the final volume), patient serum (2% of the final volume) and FBS (10% of the final volume) supplemented with 10 mL of C-BASE solution. The cells were grown for 15 days in 75 cm² flasks placed in an incubator with 5% CO₂ at a temperature of 37° C. After 2 days of incubation, the medium is substituted with 15 mL of C-BASE solution. When the cells reach 80% confluence, they are detached by means of trypsin-EDTA (5 minutes at a temperature of 37° C.); on completion of incubation, 5 mL of pure FBS and 5 mL of unsupplemented DMEM-LG medium (Gibco; Grand Island, N.Y.) are added; the detached cells in suspension are harvested, washed twice by centrifugation at 160 g for 7 minutes at 37° C. in unsupplemented DMEM-LG medium (Gibco; Grand Island, N.Y.) alone. The cells contained in the pellet thus obtained are resuspended in C-BASE solution at a concentration of 1×10⁶.

### Chondrocytes

Samples and controls are tested to verify the presence of active cartilage-producing chondrocytes at days 7, 10, 15, 30, 45, 60, 75, and 90 of culture, using the colourimetric method with Alcian blue described hereinafter.

### Isolation of Peripheral Blood Monocytes

Having withdrawn 40 mL of peripheral blood (six 7 mL EDTA tubes), the lympho-monocyte population is prepared as follows. The whole blood is split into two 20 mL aliquots and each aliquot is layered dropwise onto 25 mL of Ficoll-Paque (Amersham Pharmacia Biotech, Uppsala, Sweden) in 50 mL tubes (Lab-Tek, Nunc, Kamstrup, Denmark). The preparation is centrifuged once more at 1800 rpm for 25 minutes and the supernatant removed leaving 1 cm, and the transparent ring of monocytes which forms after the final centrifugation collected. The transparent ring thus obtained is diluted in 10 mL of RPMI 1640 (Life Technologies, Grand Island, N.Y., USA) and centrifuged at 1300 rpm for 10 minutes. The supernatant is then removed and the remainder washed a further twice by centrifugation at 1000 rpm for 10 minutes, and finally, the pellet is resuspended in 10 mL of RPMI 1640 with 10% FBS (Foetal Bovine Serum). The cells thus obtained are counted by means of a Burker chamber in order to obtain a final suspension of 5×10⁵ cells/mL. At this point the cells are seeded into Petri cells, 100 mm diameter plates (Falcon, Becton Dickinson, Labware Europe, Milan, Italy). The samples are incubated for 30 minutes at 37° C. with 5% CO₂. The time required for the monocytes obtained to adhere to the plate is thirty minutes. The cells are not left for longer periods of time in order to prevent the lymphocytes also adhering. The cells were washed, resuspended and grown as described in detail below.

Monocytoid Cell Line and Monocytes Isolated from Peripheral Blood

The cells were washed 3 times by centrifugation at 160 g for 10 minutes at room temperature in RPMI 1640 medium (Life Technologies, Grand Island, N.Y., USA) and resuspended in 15 cm plates (Lab-Tek chamber slides, Nunc, Kamstrup, Denmark) at a final concentration of 1×10⁶ cells/mL in medium composed as follows:

100 units/mL penicillin
100 μg/mL streptomycin
160 mg/L gentamycin (Schering-Plough, Milan, Italy)
2 mM L-glutamine (Life Technologies; growth medium)
50 ng/mL M-CSF (macrophage colony stimulating factor, Peprotech Inc., NJ, USA)
1000 units/mL LIF (leukaemia inhibitory factor, Santa Cruz Biotechnology, CA, USA)
1000 units/mL IL-2 (human recombinant interleukin 2)
3 nM phorbol-12-myristate-13-acetate (PMA, Santa Cruz Biotechnology, CA, USA).

The substances, weighed-out as required for the composition, were diluted in C-BASE composition, to give 1 litre of final solution.

Two types of controls were prepared: One negative control (1) of untreated cells and one control of cells treated with LIF (anti-leukaemic factor, leukaemia inhibitory factor), with M-CSF (macrophage colony stimulating factor, Peprotech Inc., NJ, USA) (2), with PMA (3 nM phorbol-12-myristate-13-acetate, Santa Cruz Biotechnology, CA, USA) and with IL-2 (recombinant human interleukin-2); as described in detail hereinafter, and in the Italian patent application TO2005A000819.

1. The control cells were washed 3 times by centrifugation at 160 g for 10 minutes at room temperature in RPMI 1640 medium (Life Technologies, Grand Island, N.Y., USA) and resuspended in 15 cm plates (Lab-Tek chamber slides, Nunc, Kamstrup, Denmark) at a final concentration of 1×10⁶ cells/mL in RPMI 1640 medium supplemented with:

### 10% FBS (Celbio, Milan, Italy)

100 units/mL penicillin
100 μg/mL streptomycin
160 mg/L gentamycin (Schering-Plough, Milan, Italy)
2 mM L-glutamine (Life Technologies; growth medium).

2. The cells were washed 3 times by centrifugation at 160 g for 10 minutes at room temperature in RPMI 1640 medium (Life Technologies, Grand Island, N.Y., USA) and resuspended in 15 cm plates (Lab-Tek chamber slides, Nunc, Kamstrup, Denmark) at a final concentration of 1×10⁶ cells/mL in RPMI 1640 medium supplemented with:

### 10% FBS (Celbio, Milan, Italy)

100 units/mL penicillin
100 μg/mL streptomycin
160 mg/L gentamycin (Schering-Plough, Milan, Italy)
2 mM L-glutamine (Life Technologies; growth medium)
50 ng/mL M-CSF (macrophage colony stimulating factor, Peprotech Inc., NJ, USA)
1000 units/mL LIF (leukaemia inhibitory factor, Santa Cruz Biotechnology, CA, USA)
1000 units/mL IL-2 (human recombinant interleukin 2)
3 nM phorbol-12-myristate-13-acetate (PMA, Santa Cruz Biotechnology, CA, USA).

All samples were incubated for 15 days in a Heraeus thermostatically controlled incubator at a temperature of 37° C. in an atmosphere containing a constant flow of 5% CO₂ (v/v in air). In all the samples, the medium was regularly changed every 7 days, leaving 20% of the medium so as not to remove all the growth-essential cytokines produced by the cells.

Samples of all the cells in the study were washed three times by centrifugation at 160 g for 10 minutes at 37° C. and subjected to cytofluorometric analysis (Epics Profile II, Coulter, Hialeath, Fla., USA) after labelling with the following anti-human mouse monoclonal antibodies (Mabs) conjugated to either R-phycoerythrin (PE) or Fluorescein-IsoThioCyanate (FITC): anti human CD14 (Santa Cruz Biotechnology, CA, USA), anti human CD34 (Santa Cruz Biotechnology, CA, USA), anti CD45 (Santa Cruz Biotechnology, CA, USA), anti c-Kit (Santa Cruz Biotechnology, CA, USA) and anti c-Met (Santa Cruz Biotechnology, CA, USA). When tested by cell cytofluorimetry (FACS), the cells are significantly positive for all the markers of stem cell expression (CD14, CD34, CD45, c-Kit, c-Met).

After the incubation period, the cells appear to be in a state of semi adhesion/suspension, with mixed oval and fibroblastoid morphologies. The cells harvested using 2% lidocaine (Sigma Aldrich, Milan, Italy) in PBS [3,4] were washed 3 times by centrifugation at 160 g for 10 minutes at 37° C. in RPMI 1640 (Life Technologies, Grand Island, N.Y.) and were incubated for a second time in accordance with the following description.

The treated controls destined to remain undifferentiated pluripotent stem cells (e.g. PSC-THP1, ICLC PD No. 05005, deposited on 18 Oct. 2005) were resuspended at a final concentration of 1×10⁵ cells per mL in 6 well plates (Lab-Tek chamber slides, Nunc, Kamstrup, Denmark) in 0.5 mL per well of final solution composed of RPMI 1640 medium supplemented with:

### 10% FBS (Celbio, Milan, Italy)

100 units/mL penicillin
100 μg/mL streptomycin
160 mg/L gentamycin (Schering-Plough, Milan, Italy)
2 mM L-glutamine (Life Technologies; growth medium)
50 ng/mL M-CSF (macrophage colony stimulating factor, Peprotech Inc., NJ, USA)
1000 units/mL LIF (leukaemia inhibitory factor, Santa Cruz Biotechnology, CA, USA)
1000 units/mL IL-2 (human recombinant interleukin 2)
3 nM phorbol-12-myristate-13-acetate (PMA, Santa Cruz Biotechnology, CA, USA).

The cells were incubated for 15 days in a Heraeus thermostatically controlled incubator at a temperature of 37° C. in an atmosphere containing a constant flow of 5% CO₂ (v/v in air) with 0.25 mL of medium being replaced every 7 days.

The monocyte and monocytoid cell samples, destined to be stimulated towards chondrocytic specialisation, were resuspended in 50 mL tubes (Lab-Tek, Nunc, Kamstrup, Denmark) at a final concentration of 2×10⁶ cells per mL in a final solution composed as follows:

100 units/mL penicillin
100 μg/mL streptomycin
160 mg/L gentamycin (Schering-Plough, Milan, Italy)
2 mM L-glutamine (Life Technologies; growth medium)
50 ng/mL M-CSF (macrophage colony stimulating factor, Peprotech Inc., NJ, USA)
1000 units/mL LIF (leukaemia inhibitory factor, Santa Cruz Biotechnology, CA, USA)
1000 units/mL IL-2 (human recombinant interleukin 2)
3 nM phorbol-12-myristate-13-acetate (PMA, (Santa Cruz Biotechnology, CA, USA).

The substances, weighed-out as required for the composition, were diluted in C-BASE PLUS composition, to give 1 litre of final solution.

The cells were incubated for 15 days in a Heraeus thermostatically controlled incubator at a temperature of 37° C. in an atmosphere containing a constant flow of 5% CO₂ (v/v in air) with 2 mL of medium being replaced every 7 days (PSC-THP1-CHONDROCYTE-LIKE cell line, chondrocytoid cell line having accession number ICLC PD No. 06001).

In particular, PSC-THP-1 cells treated for 15 days with C-BASE PLUS solution (PSCTHP-1-CHONDROCYTE-LIKE, chondrocytic cell line, having accession number ICLC PD No. 06001, deposited on 21 Mar. 2006, derived from the monocytoid stem cell line PSC-THP-1, having accession number ICLC PD No. 05005, deposited on 18 Oct. 2005). It should be noted that the cells that were induced to differentiate towards the chondrocytic phenotype, deposit matrix and stain with Alcian blue.

Furthermore, the cells were seeded on trophic cartilage tissue cultures actively producing matrix enclosed in fibrin sponge fragments (Dental Green S.r.l., Baxter, Turin, Italy). Seeding occurred by adding 5 mL of the above-described solution, containing a final concentration of 2×10⁶ cells/mL, to 60 mm diameter plates (Falcon, Becton Dickinson Labware Europe, Milan, Italy) previously prepared with cartilage tissue enclosed in fibrin sponge fragments (Dental Green S.r.l., Baxter, Turin, Italy).

The cells were incubated for 15 days in a Heraeus thermostatically controlled incubator at a temperature of 37° C. in an atmosphere containing a constant flow of 5% CO₂ (v/v in air) with 2 mL of culture medium being replaced every 7 days. Three-dimensional cartilage tissue structures were visible at the end of the 15 day culture period. Cells induced to differentiate towards the chondrocytic phenotype, appear to be ordered three-dimensionally, and actively deposit matrix which stains with Alcian blue.

### Isolation and Culture of Mesenchymal Pluripotent Stem Cells

Mesenchymal pluripotent stem cells (MSCs, mesenchymal stem cells) were extracted from samples of bone marrow (BM) taken from the femoral head (spongy tissue and bone marrow) during complete hip-replacement surgery. The nucleated cells were separated by means of a density gradient (Ficoll-Paque, Amersham) and resuspended in low-glucose Dulbecco's modified Eagle's medium (DMEM-LG, Gibco, Grand Island, N.Y., USA) supplemented with 10% foetal bovine serum (FBS, Sigma), 10 U/mL penicillin G, and 40 μg/mL gentamycin. After 24 hours in culture, the medium, along with any cells in suspension, were removed by aspiration and fresh medium added onto the adhered cells. The cells were grown in 75 cm² flasks in an incubator with 5% CO₂ at a temperature of 37° C. When the cells reach 80% confluence, they are detached using trypsin-EDTA (5 minutes incubation at 37° C. followed by the addition of 5 mL of pure FBS and 5 mL of unsupplemented DMEM-LG medium (Gibco; Grand Island, N.Y.), and the detached cells in suspension are harvested), washed twice by centrifugation at 160 g for 7 minutes at 37° C. in unsupplemented DMEM-LG medium (Gibco; Grand Island, N.Y., USA) alone. The cells in the pellets thus obtained are resuspended at a concentration of 1×10⁶ in fresh DMEM-LG medium (Gibco; Grand Island, N.Y., USA) supplemented with 10% foetal bovine serum (FBS, Sigma), 10 U/mL penicillin G, and 40 μg/mL gentamycin. The cells are reseeded after 1:4 dilution (to a final concentration 2.5×10⁴). In this study, cells after the third passage in continuous culture were used.

The MSC samples, obtained as described above, destined to be stimulated towards chondrocytic specialisation were grown on 60 mm diameter plates (Falcon, Becton Dickinson Labware Europe, Milan, Italy) for 15 days in 2 mL of original culture medium (described above), 8 mL of a final solution composed as follows:

DMEM-LG (Gibco; Grand Island, N.Y.) supplemented with 10% foetal bovine serum (FBS, Sigma), 10 U/mL penicillin G, and 40 μg/mL gentamycin, the substances, weighed-out as required for the composition, were diluted in C-BASE PLUS composition, to give 1 litre of final solution.

Cells were incubated in 60 mm diameter plates (Falcon, Becton Dickinson Labware Europe, Milan, Italy) with fragments of human cartilage (taken from the same donor as the bone marrow for the MSCs) and fragments of fibrin sponge (Dental Green S.r.l., Baxter, Turin, Italy).

The controls destined to remain undifferentiated pluripotent stem cells were grown for 15 days in 60 mm diameter plates (Falcon, Becton Dickinson Labware Europe, Milan, Italy) in 10 mL of original culture medium (fresh DMEM-LG medium, Gibco; Grand Island, N.Y., USA) supplemented with 10% foetal bovine serum, Sigma, 10 U/mL penicillin G, and 40 μg/mL gentamycin.

All cells were incubated for 15 days in a Heraeus thermostatically controlled incubator at a temperature of 37° C. in an atmosphere containing a constant flow of 5% CO₂ (v/v in air) with 2 mL of culture medium being replaced every 7 days.

### Isolation and Culture of Synovial Cells

Synovial cells (SynCs) were obtained from patients during exeresis and curettage of the synovial membrane inside the femoral-tibial joint cavity. Fresh synovial tissue sections were disaggregated in collagenase (1 mg/mL per mL of medium, Sigma) for two hours at 37° C. The pellet, washed twice in DMEM-LG (Gibco; Grand Island, N.Y., USA), free of any supplements, by centrifugation at 160 g for 10 minutes and then resuspended in complete low-glucose Dulbecco's modified Eagle's medium ([DMEM-LG] GIBCO; Grand Island, N.Y., USA) supplemented with 10% foetal bovine serum ([FBS] Sigma), 10 U/mL penicillin G, and 40 μg/mL gentamycin, is finally seeded into 75 cm² flasks and incubated in 5% CO₂ at a temperature of 37° C. After 24 hours, the medium, along with the cells in suspension, was aspirated off and fresh medium (DMEM-LG, Gibco; Grand Island, N.Y., USA, supplemented with 10% FBS, Sigma, 10 U/mL penicillin G, and 40 μg/mL gentamycin) was added over the adhered cells. The cells were grown in 75 cm² flasks in an incubator with 5% CO₂ at a temperature of 37° C. When the cells reach 80% confluence, they are detached using trypsin-EDTA (5 minutes incubation at 37° C. followed by the addition of 5 mL of pure FBS and 5 mL of unsupplemented DMEM-LG medium (Gibco; Grand Island, N.Y.), and the detached cells in suspension are harvested), washed twice by centrifugation at 160 g for 7 minutes at 37° C. in unsupplemented DMEM-LG medium (Gibco; Grand Island, N.Y., USA) alone. The cells in the pellets thus obtained are resuspended at a concentration of 1×10⁶ in fresh DMEM-LG medium (Gibco; Grand Island, N.Y., USA) supplemented with 10% foetal bovine serum ([FBS] Sigma), 10 U/mL penicillin G, and 40 μg/mL gentamycin. The cells are reseeded after 1:4 dilution (final concentration of 2.5×10⁴). In this study, cells after the second passage in continuous culture were used.

The SynC samples, obtained as described above, destined to be stimulated towards chondrocytic specialisation were grown in 60 mm diameter plates (Falcon, Becton Dickinson Labware Europe, Milan, Italy) for 15 days in 2 mL of original culture medium (described above), 8 mL of a final solution composed as follows:

DMEM-LG (Gibco; Grand Island, N.Y.) supplemented with 10% foetal bovine serum (FBS, Sigma), 10 U/mL penicillin G, and 40 μg/mL gentamycin, the substances, weighed-out as required for the composition, were diluted in C-BASE PLUS composition, to give 1 litre of final solution.

Cells were incubated in 60 mm diameter plates (Falcon, Becton Dickinson Labware Europe, Milan, Italy) with fragments of human cartilage (taken from the same donor as the SynCs) and fragments of fibrin sponge (Dental Green S.r.l., Baxter, Turin, Italy).

The controls destined to remain undifferentiated pluripotent stem cells were grown for 15 days on 60 mm diameter plates (Falcon, Becton Dickinson Labware Europe, Milan, Italy) in 10 mL of original culture medium (fresh DMEM-LG medium, Gibco, Grand Island, N.Y., USA supplemented with 10% foetal bovine serum, Sigma, 10 U/mL penicillin G, and 40 μg/mL gentamycin).

The cells were incubated for 15 days in a Heraeus thermostatically controlled incubator at a temperature of 37° C. in an atmosphere containing a constant flow of 5% CO₂ (v/v in air) with 2 mL of culture medium being replaced every 7 days.

### Preliminary Cell Suspensions

After incubation and stimulation towards assuming the chondrocytic phenotype, as described above, cell suspensions were obtained from all study samples.

All samples of the monocytoid cell lines and the semi-adhered peripheral blood monocytes were incubated for 5-8 minutes with 2% lidocaine (Sigma Aldrich, Milan, Italy) in PBS by pipetting the suspension up and down in the well, and the solution thus obtained was collected, as described in references [3,4]. The cells were washed three times by centrifugation at 160 g for 10 minutes at 37° C. with unsupplemented RPMI 1640 (Life Technologies, Grand Island, N.Y.).

Upon completion of the above-described incubation, the confluent MSC and SynCs cells are detached using trypsin-EDTA (5 minutes at 37° C.; followed by the addition of 5 mL of pure FBS and the detached cells in suspension harvested), washed twice by centrifugation at 160 g for 7 minutes at 37° C. in unsupplemented DMEM-LG medium (Gibco; Grand Island, N.Y.) alone.

The cells obtained from the pellets of the samples and corresponding controls were resuspended in 15 mL tubes (Lab-Tek chamber slides, Nunc, Kamstrup, Denmark) at a final concentration of 5×10⁵ cells/mL for subsequent phenotypic analysis (Western Blotting, direct immunofluorescence and FACS).

### Western Blotting

Western blotting first employees denaturing electrophoresis (SDS-PAGE) in order to separate the various proteins according to molecular mass, by cancelling out the charges on the amino acids which might influence migration. The cell samples, suspended in lysis buffer (1% SDS, 30 mM Tris pH 6.8, 5% glycerol) to which protease inhibitors (Protease Inhibitor Cocktail, Calbiochem, San Diego, Calif., USA) were added, were incubated for 30 minutes at 4° C. The lysates thus obtained was centrifuged at 12,000 rpm for 20 minutes at 4° C. and the supernatants collected; the protein concentrations of the samples were measured using the Bio-Rad method (Benchmark Plus assay, Bio-Rad). Prior to electrophoresis, the samples were boiled for 5 minutes in the presence of beta-mercaptoethanol and bromophenol blue. The samples were subjected to electrophoresis on a 12% gel (SDS-PAGE) and then transferred onto a PVDF membrane (Perkin Elmer Inc.). The membranes were saturated with methanol at room temperature and subsequently incubated overnight, at 4° C., with the following primary antibodies diluted 1:500 in PBS with 5% skimmed milk powder: anti-CD 34 (Santa Cruz Biotechnology, CA, USA), anti-CD 14 (Santa Cruz Biotechnology, CA, USA), anti-CD 44 (Santa Cruz Biotechnology, CA, USA), anti-CD 45 (Santa Cruz Biotechnology, CA, USA), anti-CD 71 (Santa Cruz Biotechnology, CA, USA), anti-CD 90 (Santa Cruz Biotechnology, CA, USA), anti-CD 29 (Santa Cruz Biotechnology, CA, USA), anti-CD 105 (Santa Cruz Biotechnology, CA, USA), anti CD 117/c-KIT (Santa Cruz Biotechnology, CA, USA), anti-endoglin (Santa Cruz Biotechnology, CA, USA), anti-type-II collagen (Santa Cruz Biotechnology, CA, USA), anti-aggrecan (Santa Cruz Biotechnology, CA, USA), anti-THY-1 (Santa Cruz Biotechnology, CA, USA). After five washes, the membranes were incubated with the corresponding secondary antibodies (1:1000) conjugated to horseradish peroxidase (HRP, SantaCruz Biotechnologies Inc., Santa Cruz, Calif., USA) for 1 hour at room temperature. The corresponding bands were revealed using chemiluminescence liquid (Super Signal Western Pico solution, Pierce Biotechnology Inc., Rockford, Ill., USA) and captured using photographic film.

### Immunofluorescence Protocol

Cells in suspension were incubated with 0.2 mM MitoTracker Red for 10 minutes at 37° C. After three washes by centrifugation at 160 g for 10 minutes at room temperature in PBS (pH 7.4), the cell pellets were resuspended in a fixing solution of 4% paraformaldehyde in RPMI 1640 at pH 7.4, 1 hour at room temperature. After three washes in PBS, the cells were resuspended in a solution of PBS and 0.1% Triton for 1 hour at 4° C. After three washes in PBS, the cells were seeded onto slide covers and the liquid allowed to evaporate-off in air. The cells were blocked with 20% normal goat serum for 1 hour and incubated with the following R-phycoerythrin (PE) or Fluorescein-Isothiocyanate (FITC) conjugated anti-human monoclonal antibodies for 30 minutes: anti-CD 34 (Santa Cruz Biotechnology, CA, USA), anti-CD 14 (Santa Cruz Biotechnology, CA, USA), anti-CD 44 (Santa Cruz Biotechnology, CA, USA), anti-CD 45 (Santa Cruz Biotechnology, CA, USA), anti-CD 71 (Santa Cruz Biotechnology, CA, USA), anti-CD 90 (Santa Cruz Biotechnology, CA, USA), anti-CD 29 (Santa Cruz Biotechnology, CA, USA), anti-CD 105 (Santa Cruz Biotechnology, CA, USA), anti CD 117/c-KIT (Santa Cruz Biotechnology, CA, USA), anti endoglin (Santa Cruz Biotechnology, CA, USA), anti-type-II collagen (Santa Cruz Biotechnology, CA, USA), anti-aggrecan (Santa Cruz Biotechnology, CA, USA), anti THY-1 (Santa Cruz Biotechnology, CA, USA). Specific controls with the corresponding isotypes were devised for each monoclonal antibody (Santa Cruz Biotechnology, CA, USA). The nuclei were stained using Hoechst solution (dilution 1:1000). The cover slips, mounted onto slides using moviol were examined by light microscopy [1-2].

### Biopsies and Prototype Solutions

All samples (cartilage tissue biopsies) were washed three times in isotonic saline containing antibiotics (100 units/mL of penicillin+100 μg/mL streptomycin+160 mg/L gentamycin) for 10 minutes at room temperature.

The biopsy samples were then dissected into three parts (two controls and one sample to be treated, for each patient) and suspended in 1×C-BASE solution in 15 cm plates (Lab-Tek chamber slides, Nunc, Kamstrup, Denmark).

Two types of controls were prepared: one negative control (1) treated only with isotonic saline and antibiotics (as described above), and one negative control (2) treated with cell culture medium:

1. the control biopsy samples were suspended in isotonic saline in 15 cm plates (Lab-Tek chamber slides, Nunc, Kamstrup, Denmark).

2. The control biopsy samples were then placed in 15 cm plates (Lab-Tek chamber slides, Nunc, Kamstrup, Denmark) in RPMI 1640 medium supplemented with:

### 10% FBS (Celbio, Milan, Italy)

100 units/mL penicillin
100 μg/mL streptomycin
160 mg/L gentamycin (Schering-Plough, Milan, Italy)
2 mM L-glutamine (Life Technologies; growth medium)

All samples were incubated in a Heraeus thermostatically controlled incubator at a temperature of 37° C. in an atmosphere containing a constant flow of 5% CO₂ (v/v in air).

All biopsy tissues used in culture constitute potential co-conditioning supports for the three-dimensional growth of the cell samples under study.

### Cartilage Matrix Staining Protocol

After three washes for 10 minutes at room temperature in PBS (pH 7.4), the samples were resuspended for one hour at room temperature in a fixing solution, containing 4% paraformaldehyde in RPMI 1640 at pH 7.4. The samples were treated with Alcian blue stain. This stain is constituted by a set of water-soluble basic polyvalent dyes. The blue colour is due to the presence of copper in the molecule. A 1% w/v alcian blue stain solution in PBS (pH 7.4) is added to a 3% solution of acetic acid (pH 2.5). Following incubation for 2 hours at room temperature, this composition stains indelibly by binding to both sulphonated and carboxylated acid mucopolysaccharides and glycoproteins (present in the matrix). Specific controls were prepared for each sample. All samples were washed 3 times with PBS (pH 7.4) for 5 minutes at room temperature and then observed under light microscopy. It is noted that in the chondrocytes, the mucoid substance making up the matrix (proteoglycans, mucopolysaccharides, collagen (types 10 and 2)) is stained blue [5].

### Results

### Staining the Cartilage Matrix Using the Alcian Blue Colourimetric Method

Fibrin sponge with chondrocytes obtained from biopsy tissue and re-established in vitro, treated with C-BASE-INFUS solution for 7 days. It is observed that the chondrocytes are depositing matrix and are clearly stained with Alcian blue, growing in overlapping layers, and in three dimensions.
Fibrin sponge with chondrocytes obtained from biopsy tissue and re-established in vitro, treated with C-BASE solution for 15 days. It is observed that the chondrocytes are depositing matrix and are clearly stained with Alcian blue, growing in overlapping layers, and in three dimensions.
Untreated peripheral blood monocyte controls (RPMI 1640 medium, 10% FBS, 100 units/mL penicillin, 100 μg/mL streptomycin, 160 mg/L gentamycin, 2 mM L-glutamine). No Alcian blue staining is observed.
Untreated control monocytes from peripheral blood pluripotent stem cells (CD14+, CD34+) (RPMI 1640 medium, supplemented with 10% FBS as described in Italian patent application N° TO2005A000819, and containing 1000 units/mL LIF, 50 ng/mL M-CSF and 3 nM phorbol-12-myristate-13-acetate, PMA). No Alcian blue staining is observed.
Cartilage tissue in co-culture with monocytes from peripheral blood pluripotent stem cells treated for 15 days with C-BASE solution. It is observed how the cells, which were induced to differentiate towards the chondrocytic phenotype, are depositing matrix and are clearly stained with Alcian blue, and are growing in overlapping layers and in three dimensions.
Monocytes from peripheral blood pluripotent stem cells treated for 15 days with C— BASE PLUS solution. It should be noted that the cells that were induced to differentiate towards the chondrocytic phenotype, are depositing matrix and stain with Alcian blue.
Untreated THP-1 cell (monocytoid cell line) controls (RPMI 1640 medium, 10% FBS, 100 units/mL penicillin, 100 μg/mL streptomycin, 160 mg/L gentamycin, 2 mM L-glutamine). No Alcian blue staining is observed.
PSC-THP1 cells (monocytoid stem cell line, having accession number ICLC PD No. 05005, deposited on 18 Oct. 2005) (RPMI 1640 medium, supplemented with 10% FBS as described in Italian patent application N° TO2005A000819, and containing 1000 units/mL LIF, 50 ng/mL M-CSF and 3 nM phorbol-12-myristate-13-acetate, PMA). No Alcian blue staining is observed.
Cartilage tissue in co-culture with PSC-THP-1 cells treated for 15 days with C-BASE solution. It is observed how the cells, which were induced to differentiate towards the chondrocytic phenotype, are depositing matrix and are clearly stained with Alcian blue, and are growing in overlapping layers and in three dimensions.
PSC-THP-1 cells treated for 15 days with C-BASE PLUS solution (PSCTHP-1-CHONDROCYTE-LIKE, chondrocytic cell line, having accession number ICLC PD No. 06001, derived from the monocytoid stem cell line PSC-THP-1, having accession number ICLC PD No. 05005, deposited on 18 Oct. 2005). It should be noted that the cells that were induced to differentiate towards the chondrocytic phenotype, are depositing matrix and stain with Alcian blue.
Cartilage tissue in co-culture with MSC cells (mesenchymal stem cells) treated for 15 days with C-BASE solution and fibrin sponge. It is observed how the cells, which were induced to differentiate towards the chondrocytic phenotype, are depositing matrix and are clearly stained with Alcian blue, and are growing in overlapping layers and in three dimensions.
Untreated control MSC cells (DMEM-LG, Gibco, Grand Island, N.Y., USA), supplemented with 10% foetal bovine serum, Sigma, 10 U/mL penicillin G, and 40 μg/mL gentamycin). No Alcian blue staining is observed.
MSC cells treated for 15 days with C-BASE PLUS solution. It is observed how the cells, which were induced to differentiate towards the chondrocytic phenotype, are depositing matrix and are clearly stained with Alcian blue, and are growing in overlapping layers and in three dimensions.
Cartilage tissue in co-culture with SynC cells (synovial stem cells) treated for 15 days with C-BASE solution and fibrin sponge. It is observed how the cells, which were induced to differentiate towards the chondrocytic phenotype, are depositing matrix and are clearly stained with Alcian blue, and are growing in overlapping layers and in three dimensions.
Untreated control SynC cells (DMEM-LG, Gibco, Grand Island, N.Y., USA), supplemented with 10% foetal bovine serum, Sigma, 10 U/mL penicillin G, and 40 μg/mL gentamycin). No Alcian blue staining is observed.
SybC cells (synovial stem cells) treated for 15 days with C-BASE PLUS solution. It is observed how the cells, which were induced to differentiate towards the chondrocytic phenotype, are depositing matrix and are clearly stained with Alcian blue, and are growing in overlapping layers and in three dimensions.

### Western Blotting

The samples were subjected to phenotypic analysis by Western blotting for the following markers: anti-CD 34 (Santa Cruz Biotechnology, CA, USA), anti-CD 14 (Santa Cruz Biotechnology, CA, USA), anti-CD 29 (Santa Cruz Biotechnology, CA, USA), anti-CD 44 (Santa Cruz Biotechnology, CA, USA), anti-CD 45 (Santa Cruz Biotechnology, CA, USA), anti-CD 71 (Santa Cruz Biotechnology, CA, USA), anti-CD 90 (Santa Cruz Biotechnology, CA, USA), anti-CD 105 (Santa Cruz Biotechnology, CA, USA), anti CD 117/c-KIT (Santa Cruz Biotechnology, CA, USA), anti-endoglin (Santa Cruz Biotechnology, CA, USA), anti-type-II collagen (Santa Cruz Biotechnology, CA, USA), anti-aggrecan (Santa Cruz Biotechnology, CA, USA), anti THY-1 (Santa Cruz Biotechnology, CA, USA). After five washes, the membranes were incubated with the corresponding secondary antibodies (1:1000) conjugated to horseradish peroxidase (HRP, SantaCruz Biotechnologies Inc., Santa Cruz, Calif., USA) for 1 hour at room temperature, as reported in tables 4 to 10 below.

### Immunofluorescence Protocol

The samples were subjected to phenotypic analysis by the indirect immunofluorescence and the cell cytofluorimetry (FACS) techniques for the markers anti-CD 34 (Santa Cruz Biotechnology, CA, USA), anti-CD 14 (Santa Cruz Biotechnology, CA, USA), anti-CD 44 (Santa Cruz Biotechnology, CA, USA), anti-CD 45 (Santa Cruz Biotechnology, CA, USA), anti-CD 71 (Santa Cruz Biotechnology, CA, USA), anti-CD 90 (Santa Cruz Biotechnology, CA, USA), anti-CD 29 (Santa Cruz Biotechnology, CA, USA), anti-CD 105 (Santa Cruz Biotechnology, CA, USA), anti-CD 117/c-KIT (Santa Cruz Biotechnology, CA, USA), anti-endogline (Santa Cruz Biotechnology, CA, USA), anti-type 11-collagen (Santa Cruz Biotechnology, CA, USA), anti-aggrecane (Santa Cruz Biotechnology, CA, USA), anti-THY-1 (Santa Cruz Biotechnology, CA, USA), conjugated with R-phycoerythrin (PE) or Fluoresceine-IsoTioCyanate (FITC) for 30 minutes. For each monoclonal antibody, specific controls with the relevant isotype were prepared (Santa Cruz Biotechnology, CA, USA).

### Characterisation of THP-1 Versus PSC-THP 1

The results relating to the expression of CD34, CD14, CD45, CD90, CD17/c-KIT and c-MET were expressed by means of a quantitative scale as follows:

TABLE 4

| Surface antigens | THP-1 | PSC-THP1 |
|---|---|---|
| CD34 | +++ | ++++ |
| CD14 | ++ | +++ |
| CD45 | ++++ | + |
| CD90 | ++ | ++++ |
| CD117/c-KIT | ++ | +++ |
| c-Met | +++ | ++++ |

Legend
−−−−− = absence of any fluorescence
+ = 1-5 fluorescent cells per optical field
++ = 6-10 fluorescent cells per optical field
+++ = 10-20 fluorescent cells per optical field
++++ = 20-50 fluorescent cells per optical field
+++++ >50 fluorescent cells per optical field

Characterisation of Monocytic Pluripotent Stem Cells from Peripheral Blood (MONO-PSC)

The results relating to the expression of CD34, CD14, CD45, CD90, CD117/c-KIT and c-MET were expressed by means of a quantitative scale as follows:

TABLE 5

| Surface antigens | Mono-PSC |
|---|---|
| CD34 | ++++ |
| CD14 | +++ |
| CD45 | ++ |
| CD117/c-KIT | +++ |
| c-Met | ++++ |

Legend
−−−−− = absence of any fluorescence
+ = 1-5 fluorescent cells per optical field
++ = 6-10 fluorescent cells per optical field
+++ = 10-20 fluorescent cells per optical field
++++ = 20-50 fluorescent cells per optical field
+++++ >50 fluorescent cells per optical field

### Characterisation of Mesenchymal Stem Cells (MSC)

The results relating to the expression of CD34, CD14, CD44, CD45, CD29, CD117/c-KIT, CD71, CD90 and CD 105 were expressed by means of a quantitative scale as follows:

TABLE 6

| Surface antigens | MSC |
|---|---|
| CD34 | −−− |
| CD14 | −−− |
| CD44 | +++ |
| CD45 | −−− |
| CD29 | +++ |
| CD117/c-KIT | + |
| CD71 | +++ |
| CD90 | ++++ |
| CD105 | +++ |

Legend
−−−−− = absence of any fluorescence
+ = 1-5 fluorescent cells per optical field
++ = 6-10 fluorescent cells per optical field
+++ = 10-20 fluorescent cells per optical field
++++ = 20-50 fluorescent cells per optical field
+++++ >50 fluorescent cells per optical field

### Characterisation of Synovial Cells (SYN)

The results relating to the expression of CD34, CD 14, CD45, CD29, CD117/c-KIT, CD71, CD90 and CD 105 were expressed by means of a quantitative scale as follows:

TABLE 7

| Surface antigens | SYN |
|---|---|
| CD34 | −−− |
| CD14 | −−− |
| CD45 | −−− |
| CD29 | −− |
| CD117/c-KIT | −−− |
| CD71 | + |
| CD90 | +++ |
| CD105 | +++ |

Legend
−−−−− = absence of any fluorescence
+ = 1-5 fluorescent cells per optical field
++ = 6-10 fluorescent cells per optical field
+++ = 10-20 fluorescent cells per optical field
++++ = 20-50 fluorescent cells per optical field
+++++ >50 fluorescent cells per optical field

Characterisation of Chondrocytic Cells from Cartilage Tissue Biopsies (Chondrocytes)

The results relating to the expression of CD34, CD14, CD45, CD29, CD117/c-KIT, CD71, type-II collagen and aggrecan were expressed by means of a quantitative scale as follows:

TABLE 8

| Surface antigens | chondrocytes |
|---|---|
| CD34 | −−− |
| CD14 | −−− |
| CD45 | −−− |
| CD29 | −− |
| CD117/c-KIT | + |
| CD71 | + |
| Type-II collagen | ++++ |
| aggrecan | +++++ |

Legend
−−−−− = absence of any fluorescence
+ = 1-5 fluorescent cells per optical field
++ = 6-10 fluorescent cells per optical field
+++ = 10-20 fluorescent cells per optical field
++++ = 20-50 fluorescent cells per optical field
+++++ >50 fluorescent cells per optical field

Characterisation of the Chondrocytic Cell Line PSCTHP-1-CHONDROCYTE-LIKE Derived from PSC-THP1

The results relating to the expression of CD34, CD14, CD45, CD29, CD117/c-KIT, CD71, CD90, type-11 collagen and aggrecan were expressed by means of a quantitative scale as follows:

TABLE 9

| | PSC-THP1 |
|---|---|
| Surface antigens | CHONDROCYTE LIKE |
| CD34 | ++ |
| CD14 | + |
| CD45 | −−− |
| CD29 | −− |
| CD117/c-KIT | ++ |
| CD71 | ++++ |
| CD90 | +++ |
| Type-II collagen | +++++ |
| aggrecan | +++++ |

Legend
−−−−− = absence of any fluorescence
+ = 1-5 fluorescent cells per optical field
++ = 6-10 fluorescent cells per optical field
+++ = 10-20 fluorescent cells per optical field
++++ = 20-50 fluorescent cells per optical field
+++++ >50 fluorescent cells per optical field

### Characterisation of the Treated Cells Versus Untreated Controls

The results relating to the expression of type-II collagen and aggrecan were expressed by means of a quantitative scale as follows:

TABLE 10

| | | | | Treated | | Treated |
|---|---|---|---|---|---|---|
| | THP-1 | Treated | MSC | MSC | Synovial | Synovial |
| Markers | control | THP-1 | control | sample | control | sample |
| Type-II | −−− | +++ | −−− | ++++ | −−− | +++ |
| collagen | | | | | | |
| aggrecan | −−− | +++ | −−− | ++++ | −−− | +++ |

Legend
−−− = absence of any bands
−/+ = slight presence of a band
+ = thin band present
++ = medium band present
+++ = broad band present
++++ = high band present
+++++ = abundant band present

Of course, without prejudice to the principle of the invention, the details of implementation and the embodiments may vary greatly with respect to what is described and illustrated herein purely by way of example, without, despite this, departing from the scope of the present invention, as defined in the following claims.

### C-Base-Infus—Clinical Study on the Dog

### Protocol

### 1. Enrolment

Twenty cases (pure-bred dogs, different sizes, body weight, sex and ages)

### 2. Inclusion Criteria

Joint alterations.

### 3. Clinical Procedure

For all of the examined dogs, the following procedures will be adopted.

### 4. Method

The amount of the inoculated solution is equal to the amount of the aspirated and analysed synovial fluid.

a. Time Zero
Gather anamnestic information and carry out a clinical examination comprising a complete hematochemical test, including lactates in order to assess metabolic oxidation;
X-Ray of the interested joint;
ROM (Range Of Motion);
Exploratory arthroscopy;
Exclusion of any intra-articular curettage;
Arthrocentesis with aspiration and morpho-cytological and biochemical examination of the synovial fluid;
Histological examination of a sample of the joint cartilage;
Intra-articular inoculation of an amount of the tested equal to the amount of the aspirated synovial fluid.

Weekly and for three/four times, depending on the severity of the initial disease pattern, synovial examination was carried out and the solution was intra-articularly re-injected.

b. End of the Treatment Cycle
Arthrocentesis with aspiration and morpho-cytological and biochemical examination of the synovial fluid;
Histological examination of a sample of the joint cartilage;
X-Ray of the interested joint;
ROM (Range Of Motion);
lactacidemia;
clinical examination and reporting.

### Notes

The weekly cytological examination of the synovial fluid allows continuous monitoring of the evolution of the disease, either towards recovery or not, linked to the ongoing therapy. Neither corticosteroids nor FANS were used in any way, in order to avoid interference with the results. The selected laboratory is authorized by Regione Piemonte, AUT.G.R. of 10 December, 1990 no. 193-2412.

### CONCLUSIONS

The product designated as “C-BASE-INFUS” used in the clinical studies carried out on 20 dogs belonging to different breeds and of different sizes, showed very good tolerability in all of the subjects, without any apparent systemic effect, independently of the age, size, sex and joint site. The ROM (Range of Motion) increased in 85% of the cases, whilst a clinical improvement of the walk was observed in 87% of the cases. Pain upon palpation decreased in 80% of the cases. Viscosity of the synovial fluid always increased, resulting in enhanced joint lubrication, indicating a clear modification of the physical parameters of the pathological microenvironment of the joint. The synovial fluid itself showed a positive development pattern, with a trend of the laboratory values towards normalization. The X-Ray showed slowing down or stabilization of DJD (Degenerative Joint Disease). It is pointed out that the proprietors of the 20 treated dogs were satisfied with the intra-articular inoculation, which is relatively easy. In our opinion, this preparation, which is apparently capable of bringing the extra-cellular microenvironment of the joint back to a physiological condition, may represent a valid innovative and interesting therapeutic alternative. It cannot be ruled out that, in the most severe cases, the use of FANS in combination with the product “C-BASE-INFUS” may be envisaged, so as to combine an analgesic pharmacological activity with a normalization activity on the microenvironment of the joint, thereby further improving prognosis. Finally, it is reasserted that the use of the preparation “C BASE INFUS” showed no signs of intolerance of any kind, and showed very good tolerability in all of the twenty dogs treated, without any apparent systemic effect.

### Discussion

Disorders of the joint cartilage can affect individuals of any age, but are particularly important when they occur in young patients leading an active life.

It is known that, contrary to the bone tissue which shows strong regeneration abilities, hyaline cartilage, which is sometimes considered the noble tissue of the musculoskeletal apparatus, is characterized by the absence of any haematic, lymphatic or nervous support, which are essential for tissue repair. As a matter of fact, only the small losses of substance are replenished by fibrocartilage tissue, whilst the bigger ones are seldom replenished: rather deep lesions (Outerbrige grade III-IV) do not undergo spontaneous healing and in the long-term they progress towards degeneration of the joint surface.

The great advance in the field of cellular biology and biotechnology led in the last years to the development of techniques aimed at the in vitro restoration of a physiological tissue microenvironment [20-23].

Our study confirms the efficacy of the procedure for the in vitro and in vivo regeneration of a physiological tissue microenvironment in the treatment of joint cartilage lesions.

The restoration of a physiological microenvironment quickly leads to the reconstitution of a normally functioning cartilage matrix. The extra-cellular activity of the C-BASE, C-BASE PLUS and C-BASE-INFUS solutions, together with the restoration of an optimal microenvironment for the residing cells, enables the cells to function properly with a physiological deposition of normal matrix (hyaline cartilage) and a functional recovery of the treated joint.

The appearance in the treated joints of hyaline cartilage only, comparable to the undamaged one, indicates an extra-cellular activity of the C-BASE-INFUS solution, which enables the residing cells to restore their normal physiological activities (deposition of normal cartilage matrix).

On the other hand, the absence of any deposition of fibrous cartilage in the joints treated with the C-BASE, C-BASE PLUS and C-BASE-INFUS solutions, which would be a typical response to a pathological damage, may provide evidence against the prevalence of an intra-cellular fibrous tissue reparation activity.

The histological in vitro and in vivo results obtained both in the short term (7 days) and upon a two months of culture with the C-BASE, C-BASE PLUS and C-BASE-INFUS solutions, confirm the formation of morphological optimal hyaline cartilage persisting over time.

The results obtained seem to indicate that C-BASE-INFUS may be considered as an optimal culture medium highly effective in maintaining the viability of the cartilage cells and in retaining their physiological functionality both in vitro and in vivo.

### BIBLIOGRAPHY

1. Tsuchiya S, et al. Int. J. Cancer. 1980; 26:171-176.
2. Rabinovitch, M. and De Stefano, M. J. J. Exp. Med. 1976; 143: 290-304.
3. McDennott A G P, et al. Clin. Orthop. 1985; 197:96-101.
4. Grande D A, et al. J. Orthop. Res. 1989; 7:208-18.
5. French M M, et al. J. Cell. Biol. 1999 May 31; 145(5):1103-15.

1. A composition capable of accelerating differentiation of stem cells into cells with a chondrocytic phenotype, the composition comprising in combination at least one growth factor, at least one proteolytic enzyme and at least one from a sugar, an amino acid, a vitamin factor, a vitamin, a nucleotide and a nucleoside, in a physiologically acceptable carrier or diluent.

2. The composition according claim 1, wherein said at least one proteolytic enzyme is selected from the group consisting of papain, collagenase serrathiopeptidase, heparanase, DNAse, elastase, bromelain, bradykinase, Clostridium peptidase, enzymes expressed by Lactobacillus acidophilus, enzymes expressed by the genus Aspergillus, proteases, alliinases, and fibrinolysin.

3. The composition according to claim 1, wherein said at least one growth factor is selected from the group consisting of TGF-B (transforming growth factor Beta), LIF (leukaemia inhibitory factor), ITS (insulin-transferrin-selenium), insulin, M-CSF (macrophage colony stimulating factor), IL-2 (Interleukin-2), PMA (phorbol-12-myristate-13-acetate), autologous serum, corticosteroids, parasympathicolytics, and glucagon.

4. The composition according to claim 1, wherein said at least one amino acid is selected from the group consisting of methionine, cystine, N-acetylcysteine, cysteine, glycine, leucine, isoleucine, proline, glutamine, arginine, glutamic acid, histidine, histidine-HCl—H₂O, lysine, lysine-HCl, phenylalanine, serine, threonine, tryptophane, tyrosine, tyrosine disodium salt, valine, proline, and hydroxyproline.

5. The composition according to claim 1, the composition further comprising at least one peptide.

6. The composition according to claim 5, wherein said at least one peptide is selected from the group consisting of glutathione, collagen, elastin, and wheat extract.

7. The composition according to claim 1, wherein said at least one sugar is selected from the group consisting of a monosaccharide, a polysaccharide, alcohol derivatives and mixtures thereof.

8. The composition according to claim 7, wherein said sugar is selected from the group consisting of glucose, sucrose, glucans, mannans, glucomannans, fucose, fructose, heparan sulphates, pectins, and starches.

9. The composition according to claim 1, the composition further comprising at least one mucopolysaccharide.

10. The composition according to claim 9, wherein said at least one mucopolysaccharide is selected from the group consisting of hyaluronic acid, and chondroitin sulphates.

11. The composition according to claim 1, wherein said at least one vitamin is selected from the group consisting of retinoic acid, retinol, ascorbic acid, pantothenic acid, D-calcium pantothenate, pyridoxine, pyridoxine-HCl, folic acid, niacinamide, riboflavin, cobalamine, para-aminobenzoic acids and biotin.

12. The composition according to claim 1, wherein said at least one vitamin factor is selected from the group consisting of inositol, myo-inositol, choline chloride, pyruvic acid, sodium pyruvate, putrescine- and putrescine-HCl.

13. The composition according to claim 1, the composition further comprising a salt.

14. The composition according to claim 13, wherein said salt comprises at least one salt selected from the group consisting of calcium gluconate, calcium phosphate, sodium bicarbonate, calcium chloride, magnesium chloride, magnesium sulphate, potassium chloride, potassium phosphate, sodium chloride, calcium nitrate, zinc chloride, ferric nitrate, D-calcium pantothenate, sodium pyruvate, and tyrosine disodium salt.

15. The composition according to claim 1, wherein said at least one proteolytic enzyme is present in an amount expressed as weight per volume with respect to the total volume, the at least one proteolytic enzyme present in an amount comprised between 1 ng/L and 2 g/L.

16. The composition according to claim 1, wherein said at least one amino acid is present in an amount expressed as percentage by volume with respect to the total volume of the composition, the at least one amino acid present in an amount comprised between 0.001% and 40%.

17. The composition according to claim 1, wherein said at least one nucleotide or nucleoside is present in an amount expressed as percentage by volume with respect to the total volume of the composition, the at least one nucleotide or nucleoside present in an amount comprised between 0.0001% and 20%.

18. The composition according to claim 5, wherein said at least one peptide is present in an amount expressed as percentage by volume with respect to the total volume of the composition, the at least one peptide present in an amount comprised between 0.001% and 40%.

19. The composition according to claim 1, wherein said at least one sugar is present in an amount expressed as percentage by volume with respect to the total volume of the composition, the at least one sugar is present in an amount comprised between 0.001% and 40%.

20. The composition according to claim 9, wherein said mucopolysaccharide is present in an amount expressed as weight per volume with respect to the total volume of the composition, the mucopolysaccharide being present in an amount comprised between 0.01 mg/L and 5 g/L.

21. The composition according to claim 1, wherein said at least one vitamin or said at least one vitamin factor is present in an amount expressed as percentage by volume with respect to the total volume of the composition, the at least one vitamin or said at least one vitamin factor being present in an amount comprised between 0.0001% and 40%.

22. The composition according to claim 1, wherein said growth factor is present in an amount expressed as weight per volume with respect to the total volume of the composition, the growth factor being present in an amount comprised between 1 ng/L and 2 g/L.

23. The composition according to claim 2, wherein said corticosteroid is present in an amount expressed as percentage by volume with respect to the total volume of the composition, the corticosteroid being present in an amount comprised between 0.0001% and 20%.

24. The composition according to claim 13, wherein said at least one salt is present in an amount expressed as weight per volume with respect to the total volume of the composition, the at least one salt being present in an amount comprised between 0.01 μg/L and 20 g/L.

25. The composition according to claim 1, wherein the composition is a cell culture medium comprising a cell culture solution as a carrier or diluent.

26. The composition according to claim 1, wherein the composition is a pharmaceutical composition or a medical device for human or veterinary use, said composition comprising a pharmaceutically acceptable carrier or diluent.

27. A method for stimulating in vitro differentiation of pluripotent stem cells into cells with a chondrocytic phenotype, and/or for restoration of an original trophism of chondrocytes from a cartilage tissue, the method comprising
administering to said pluripotent stem cells and/or chondrocytes an effective amount of the composition according to claim 1.

28. A medicament capable of stimulating differentiation of pluripotent stem cells into cells with a chondrocytic phenotype, and/or for restoration of an original trophism of chondrocytes from a cartilage tissue, the medicament comprising an effective amount of the composition according to claim 1.

29. A medical device capable of stimulating in situ differentiation of pluripotent stem cells into cells with a chondrocytic phenotype, and/or for restoration of an original trophism of chondrocytes from a cartilage tissue, said device configured for administering a composition to a pathological site wherein proteolytic enzyme-producing pro-inflammatory cells are present, the composition comprising in combination at least one sugar one amino acid and at least one vitamin factor or a vitamin in a physiologically acceptable vehicle or diluent.

30. A method for in vitro differentiation of pluripotent stem cells into cells with a chondrocytic phenotype, the method comprising the steps of:
i) providing pluripotent stem cells;
ii) providing a culture medium according to claim 25;
iii) culturing said pluripotent stem cells in said culture medium, so as to obtain cells with a chondrocytic phenotype.

31. The method according to claim 30, wherein said culture medium comprises at least one of a macrophage colony stimulating factor and a leukaemia inhibitory factor.

32. The method according to claim 30, wherein said culture medium comprises at least one interleukin.

33. The method according to claim 30, wherein said culture medium comprises at least one antibiotic.

34. The method according to claim 30, wherein culturing said pluripotent stem cells in said culture medium is carried out in the presence of a scaffold.

35. The method according to claim 34, wherein said scaffold is selected from the group consisting of fibrin, collagen, cartilage fragments, alginates, hyaluronic acid, hyaluronate, polyglycolic acid, hydroxyapatite, and carboxy methyl cellulose modified with amide groups on the polymeric chains.

36. The method according to claim 30, wherein said pluripotent stem cells are native or non-native adult human and/or animal pluripotent stem cells of hematopoietic or mesenchimal origin.

37. The method according to claim 30, in that wherein said pluripotent stem cells are the cell line PSC-THP1 deposited on 18 Oct. 2005 with the Advanced Biotechnology Centre, Interlab Cell Line Collection, Genoa, Italy, under the accession number ICLC PD No. 5005.

38. A derived cell line comprising cells having a chondrocytic phenotype obtained by the method according to claim 30, which is the cell line PSC-THP1-CHONDROCYTE-LIKE deposited on 21 Mar. 2006 with the Advanced Biotechnology Centre, Interlab Cell Line Collection, Genoa, Italy, under the accession number ICLC PD N° 06001.

39. A medicament capable of stimulating differentiation in vivo of pluripotent stem cells into cells having a chondrocytic phenotype and/or restoration of an original trophism of chondrocytes from a cartilage tissue, the medicament comprising cells obtainable in a culture medium by the method according to claim 30.

40. The medicament according to claim 39, wherein said culture medium further comprises chondrocyte matrix.

41. The medicament according to claim 39, wherein said medicament is capable of being administered by endoarticular infiltration.

42. A method for producing a chondrocyte matrix comprising the step of culturing the derived cell line having a chondrocytic phenotype according to claim 38.

43. The method according to claim 42, wherein said derived cell line is cultured in presence of a growth factor.

44. The method according to claim 43, wherein said growth factor is used in an amount expressed as percentage weight with respect to the total volume of the composition, the growth factor used in an amount comprised between 0.01 ng/L and 2001/L.

45. A kit of parts comprising at least one proteolytic enzyme, at least one growth factor and at least one of a sugar, an amino acid, a vitamin factor, a vitamin, a nucleotide and a nucleoside for simultaneous, sequential or separate use in cartilage regeneration therapy.

46. The kit of parts according claim 45, wherein said at least one proteolytic enzyme is selected from the group consisting of papain, collagenase serrathiopeptidase, heparanase, DNAse, elastase, bromelain, bradykinase, Clostridium peptidase, enzymes expressed by Lactobacillus acidophilus, enzymes expressed by the genus Aspergillus, proteases, alliinases and fibrinolysin.

47. The kit of parts according to claim 45, wherein said at least one amino acid is selected from the group consisting of methionine, cystine, N-acetylcysteine, cysteine, glycine, leucine, isoleucine, proline, glutamine, arginine, glutamic acid, histidine, histidine-HCl—H₂O, lysine, lysine-HCl, phenylalanine, serine, threonine, tryptophane, tyrosine, tyrosine disodium salt, valine, praline and hydroxyproline.

48. The kit of parts according to claim 45, wherein said at least one growth factor is selected from the group consisting of transforming growth factor beta, leukaemia inhibitory factor, insulin-like growth factor, insulin-transferrin-selenium, insulin, macrophage colony stimulating factor, interleukin-2, phorbol-12-myristate-13-acetate, and autologous serum.

49. The kit of parts according to claim 45, wherein said growth factor is in association with a sugar and/or a salt.

50. The kit of parts according to claim 49, wherein said sugar is selected from the group consisting of glucose, sucrose, glucans, mannans, glucomannans, fucose, fructose, heparan sulphates, pectins, and starches.

51. The kit of parts according to claim 49, wherein said salt is selected from the group consisting of calcium gluconate, calcium phosphate, sodium bicarbonate, calcium chloride, magnesium chloride, magnesium sulphate, potassium chloride, potassium phosphate, sodium chloride, calcium nitrate, zinc chloride, ferric nitrate, D-calcium pantothenate, sodium pyruvate, and tyrosine disodium salt.

52. A method for stimulating differentiation of pluripotent stem cells into cells with a chondrocytic phenotype in an individual, and/or for restoration of an original trophism of chondrocytes from a cartilage tissue in the individual, the method comprising administering to the individual an effective amount of the composition according to claim 1.

53. The composition according claim 2, wherein the collagenase is selected from the group consisting of collagenase type-IA, collagenase type-II, and collagenase type-IV.

54. The composition according to claim 5, wherein the at least one peptide is an oligopeptide or a tripeptide.

55. The composition according to claim 1, wherein said at least one proteolytic enzyme is present in an amount expressed as weight per volume with respect to the total volume, the at least one proteolytic enzyme present in an amount comprised between 0.01 mg/L and 20.0 mg/L.

56. The composition according to claim 1, wherein said at least one amino acid is present in an amount expressed as percentage by volume with respect to the total volume of the composition, the at least one amino acid present in an amount comprised between 0.01% and 20%.

57. The composition according to claim 1, wherein said at least one nucleotide or nucleoside is present in an amount expressed as percentage by volume with respect to the total volume of the composition, the at least one nucleotide or nucleoside present in an amount comprised between 0.01% and 2%.

58. The composition according to claim 5, wherein said at least one peptide is present in an amount expressed as percentage by volume with respect to the total volume of the composition, the at least one peptide present in an amount comprised between 0.01% and 20%.

59. The composition according to claim 1, wherein said at least one sugar is present in an amount expressed as percentage by volume with respect to the total volume of the composition, the at least one sugar is present in an amount comprised between 0.01% and 20%.

60. The composition according to claim 9, wherein said mucopolysaccharide is present in an amount expressed as weight per volume with respect to the total volume of the composition, the mucopolysaccharide present in an amount comprised between 0.1 mg/L and 200 mg/L.

61. The composition according to claim 1, wherein said at least one vitamin or said at least one vitamin factor is present in an amount expressed as percentage by volume with respect to the total volume of the composition, the at least one vitamin or said at least one vitamin factor present in an amount comprised between 0.001% and 20%.

62. The composition according to claim 1, wherein said growth factor is present in an amount expressed as weight per volume with respect to the total volume of the composition, the said growth factor present in an amount comprised between 1 ug/L and 20 mg/L.

63. The composition according to claim 2, wherein said corticosteroid is present in an amount expressed as percentage by volume with respect to the total volume of the composition, the corticosteroid present in an amount comprised between 0.0001% and 1%.

64. The composition according to claim 13, wherein said at least one salt is present in an amount expressed as weight per volume with respect to the total volume of the composition, the at least one salt present in an amount comprised between 10 μg/L and 10 g/L.

65. The method according to claim 33, wherein the at least one antibiotic is selected from the group consisting of gentamycin, penicillin and streptomycin.

66. The method according to claim 43, wherein said growth factor is used in an amount expressed as percentage weight with respect to the total volume of the composition, the growth factor used in an amount comprised between 1 ng/L and 200 mg/L.
